(19) United States
(12) Patent Application Publication
(10) Pub. No.: US 2023/0047034 A1
(43) Pub. Date: Feb. 16, 2023

(54) INFUSION PUMPS AND METHODS WITH SHAPE MEMORY WIRE DRIVEN SYRINGE MECHANISM

(71) Applicant: Tandem Diabetes Care, Inc., San Diego, CA (US)

(72) Inventor: Michael Michaud, San Diego, CA (US)

(21) Appl. No.: 17/886,998

(22) Filed: Aug. 12, 2022

Related U.S. Application Data
(60) Provisional application No. 63/232,300, filed on Aug. 12, 2021.

(51) Int. Cl. A61M 5/172; A61M 5/142

(52) U.S. Cl. CPC A61M 5/172; A61M 5/14244; A61M 2205/0266; A61M 2005/14506

(57) ABSTRACT

Disclosed herein are apparatuses and methods for a user-wearable infusion pump actuated with a shape memory alloy (SMA) wire. Embodiments enable an SMA wire driven infusion pump using a single length of SMA wire to provide multiple pulse sizes and corresponding medicament dispensing sizes. The multiple pulse sizes can include a larger pulse size for larger volumes to limit battery drain but smaller pulse sizes for precision of delivery.

### RELATED APPLICATION

The present application claims the benefit of U.S. Provisional Application No. 63/232,300, filed Aug. 12, 2021, which is hereby incorporated herein in its entirety by reference.

### FIELD OF THE INVENTION

The present invention relates generally to ambulatory infusion pumps and, more particularly, to a user-wearable pump, such as a patch pump, for delivering medicament such as insulin to a patient.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps are configured as portable or ambulatory infusion devices can provide continuous subcutaneous insulin injection and/or infusion therapy as an alternative to multiple daily injections of insulin via a syringe or an injector pen. Such ambulatory infusion pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

One type of pump that has been developed is a patch pump, or micro pump. Patch pumps generally are small pumps, typically ambulatory, that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament.

Ambulatory infusion pumps can employ various actuation mechanisms for driving the system to deliver medicament to the user, including electromagnetic drive motors, piezoelectric motors, and electrically driven shape-memory alloy (SMA) wire actuators. With regard to SMA wire, using SMA wire has been used in a variety of miniaturized mechanism designs, including patch pumps, and has cost and size advantages over other actuation mechanism.

Such SMA actuators operate by heating the SMA wire to transition the wire from a first configuration to a second configuration to drive a plunger to deliver medicament. One of the characteristics of electrically driven SMA wire actuators is that high current is required to heat the wire to generate the strain used to actuate the mechanism. Some low cost batteries which are suitable for disposable medicament devices, such as patch pumps, are not optimized for high current short duration duty cycles and exposing them to this duty cycle reduces usable capacity compared to duty cycles that have high duration low current duty cycles. For this reason, it is desirable to minimize the number of high current short duration cycles required for a given application such as a patch pump that uses SMA wire and these types of batteries. For an application like diabetes that generally delivers a dose every five minutes often of a size that requires more than one pulse, a series of pulses is regularly needed that can quickly drain the batteries.

### SUMMARY

Disclosed herein are apparatuses and methods for a user-wearable infusion pump actuated with a shape memory alloy (SMA) wire. Embodiments enable an SMA wire driven infusion pump using a single length of SMA wire to provide multiple pulse sizes and corresponding medicament dispensing sizes. The multiple pulse sizes can include a larger pulse size for larger volumes to limit battery drain but smaller pulse sizes for precision of delivery.

In an embodiment, a user-wearable infusion pump can include a housing, a reservoir configured to contain a medicament disposed in the housing and a delivery mechanism configured to deliver medicament from the reservoir to a user. An actuation wire can be configured to be energized to actuate the delivery mechanism to deliver medicament from the reservoir to a user.

In an embodiment, user-wearable infusion pump can include a housing, a reservoir configured to contain a medicament disposed in the housing and a delivery mechanism configured to deliver medicament from the reservoir to a user. The delivery mechanism can be configured to provide two different predetermined medicament dispense sizes.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIGS. 1A-1C depict various views of a patch pump according to an embodiment of the disclosure.

FIGS. 2A-2G depict various internal views of the patch pump of FIGS. 1A-1C.

FIGS. 3A-3C schematically depict the patch pump of FIGS. 1A-1C and FIGS. 2A-2G.

FIGS. 4A-4B depict remote control devices for a pump system according to the disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

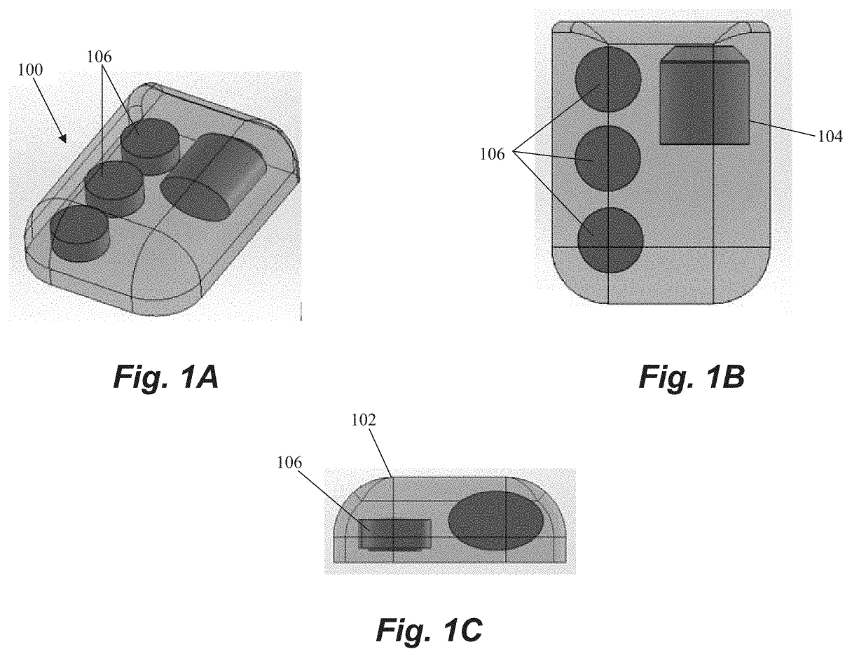

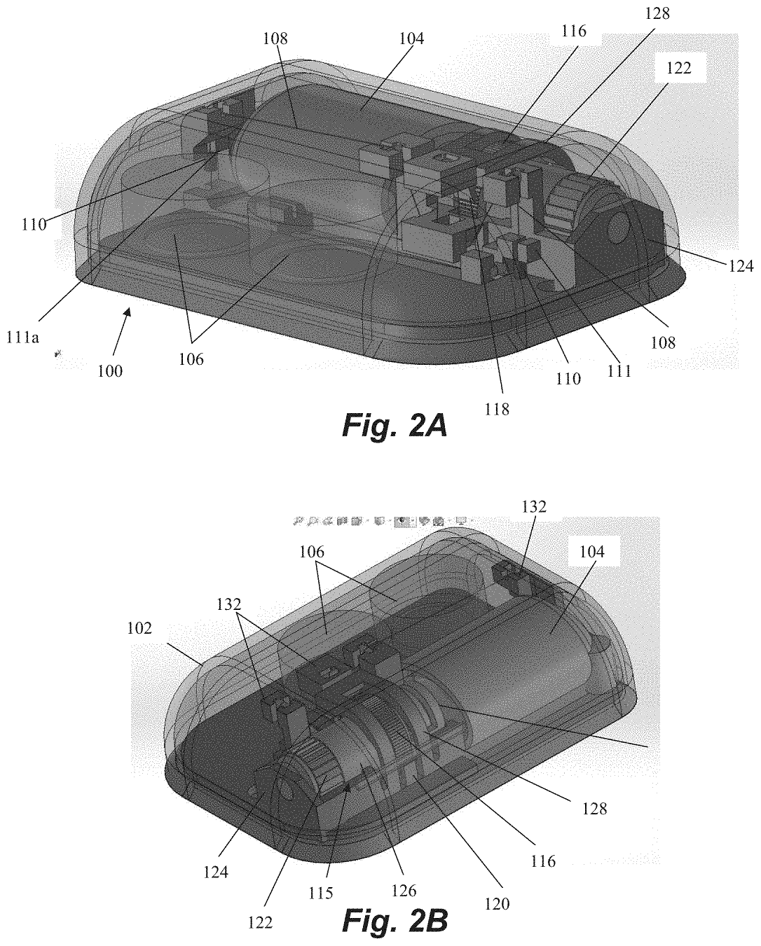

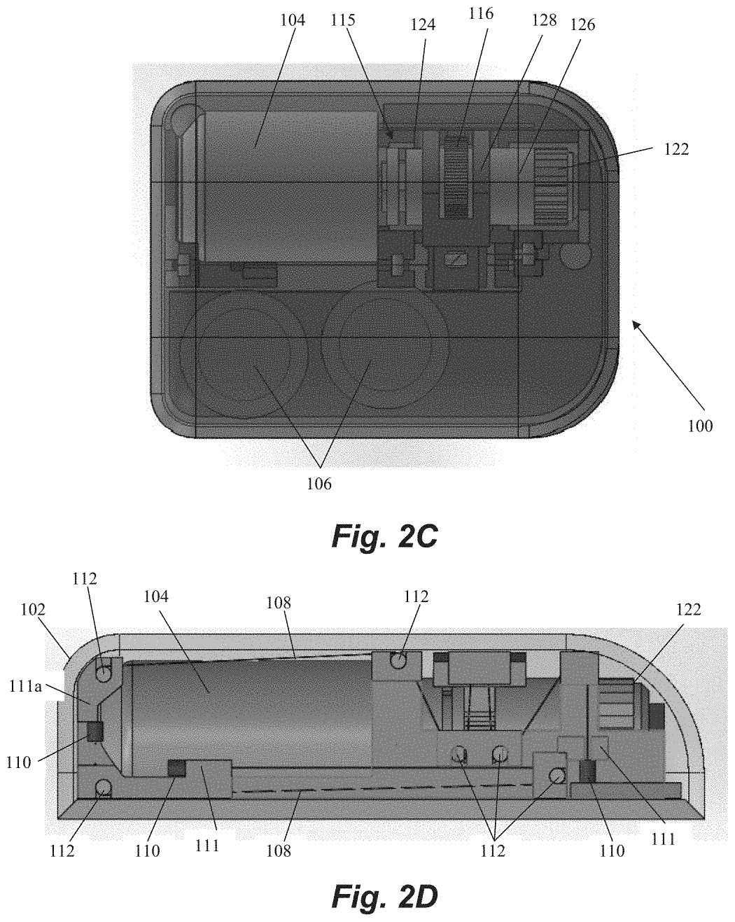

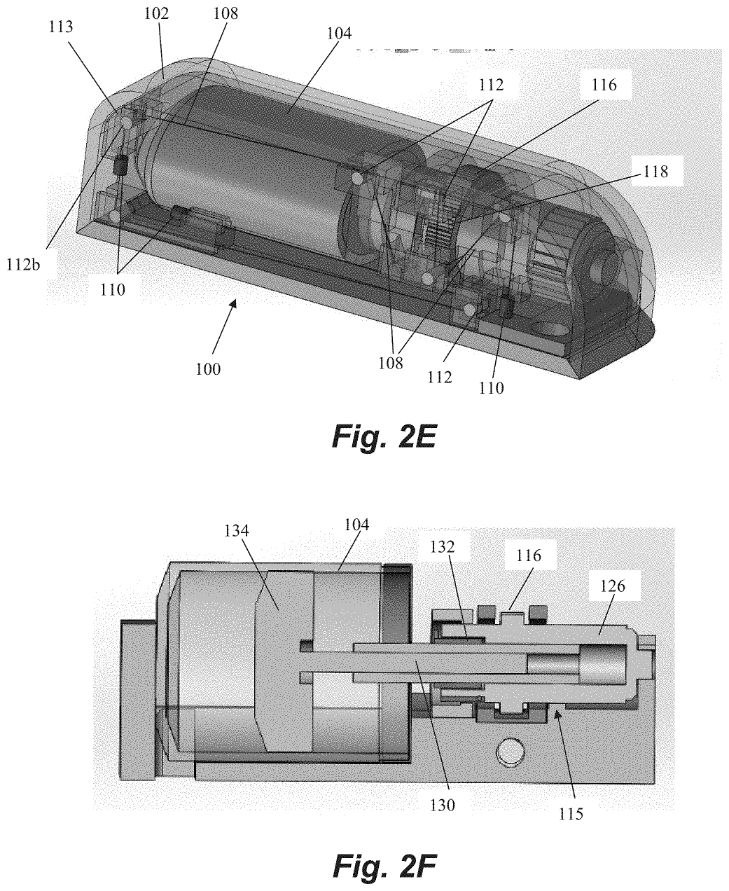

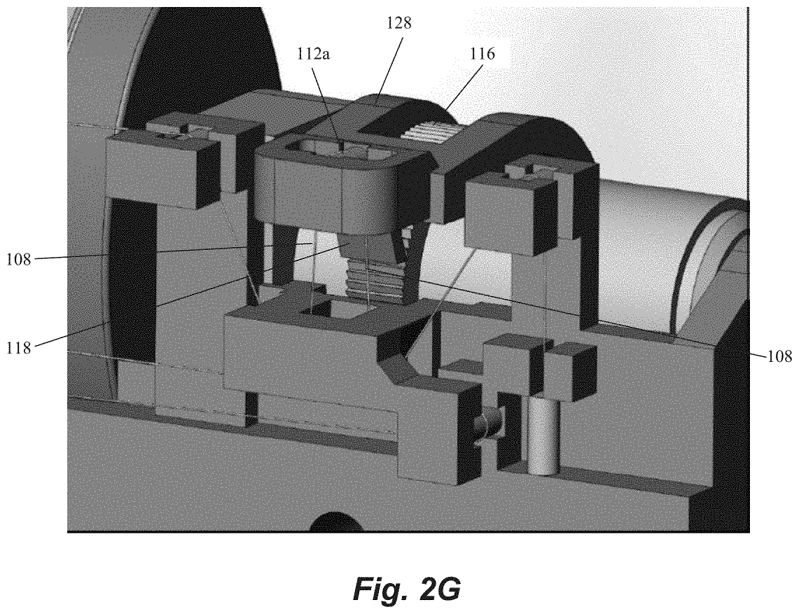

FIGS. 1A-1C and 2A-2G depict a user-wearable infusion pump that can be configured as a disposable patch pump 100 according to the disclosure. Patch pump 100 can include a housing 102 containing a reservoir 104 configured to contain a medicament. In embodiments, patch pump 100 can be a single use ambulatory pump intended to be worn directly on a user's body and disposed of after using the medicament in the reservoir 104. In some embodiments, the reservoir 104 can have a small volume, i.e., 1 mL or less, and be intended for use with concentrated insulin and/or for pediatric applications that have small delivery volumes. Such a patch pump 100 can also include an adhesive patch (not pictured) that enables the user to wear the pump directly on the user's body. Patch pump 100 can be powered by one or more coin cell batteries 106 disposed within housing 102. The components of the system can be mounted on a printed circuit board assembly (PCBA) electrically connecting the battery with the other electronic components of the system.

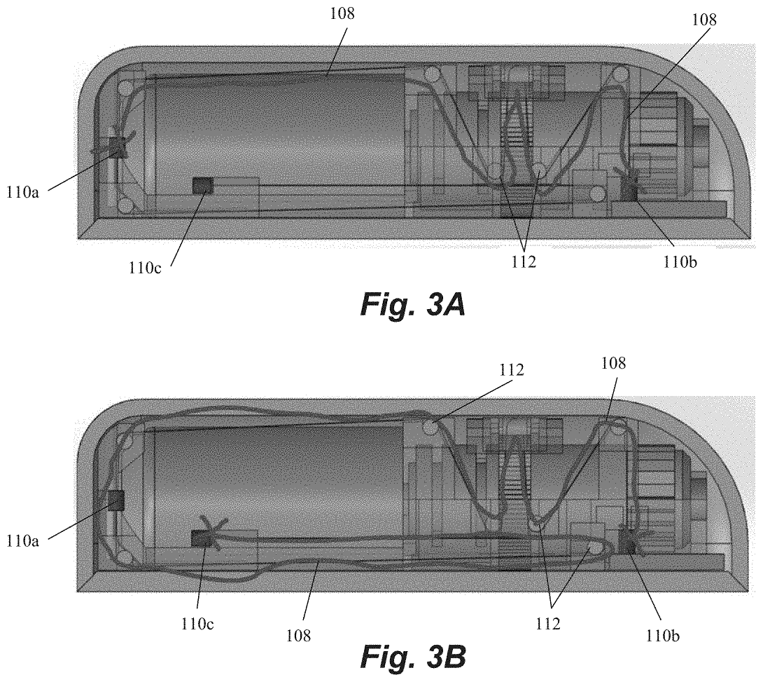

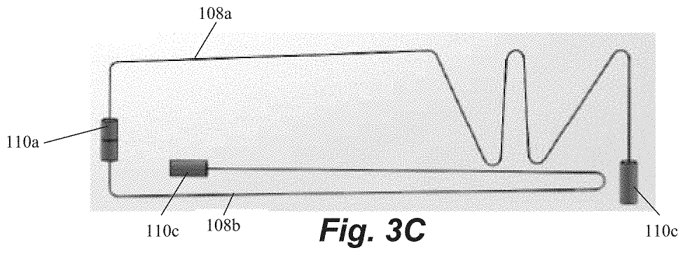

A shape memory alloy (SMA) wire 108, such as Nitinol, can be crimped to and extend from a plurality of crimp connectors 110 and be routed around a plurality of pulleys 112 within housing 102. In embodiments, SMA wire 108 can be configured as a single continuous wire. Referring to FIGS. 2D-2E, 2G and 3A-3C, in the depicted embodiment the pump can include three crimp connectors 110 and eight pulleys 112 that, as will be discussed in more detail below, can define two distinct electrical paths for SMA wire 108. When SMA wire 108 is heated using energy from the battery 106, the wire 108 transitions from a first length to a second, shorter length to actuate a delivery mechanism 115 to deliver medicament from the reservoir to the user through a cannula (not pictured) extending through an opening in the housing 102 and into the skin of the user beneath the pump 100. The crimp connectors 110 can be prevented or limited from moving when the SMA wire shortens by one or more crimp stops 111.

A delivery mechanism frame 124 can be disposed around delivery mechanism 115 to hold the delivery mechanism in place and axially constrain the delivery mechanism. The delivery mechanism 115 of pump 100 includes a drive gear 116 that is rotated when SMA wire 108 is actuated and a syringe body 126. Delivery mechanism frame 124 can include a flexible member 128 extending around syringe body 126 and along both sides of drive gear 116 to define a living hinge that provides the actuation force. Referring in particular to FIG. 2G, a pulley 112a can be disposed within an opening in flexible member 128. When the SMA wire 108 shortens, the wire 108 pulls down on pulley 112a, which in turn pulls down on flexible member 128. Flexible member 128 includes a drive tooth 118 that interfaces with the teeth of drive gear 116 to ensure precise rotation of drive gear 116. When the flexible member 128 is pulled down by SMA wire 108, the drive tooth 118 accordingly moves down and provides a force that rotates the drive gear 116 a predetermined amount.

Referring now to FIG. 2F, when drive gear 116 is rotated, syringe body 126 of delivery mechanism 115 also rotates. Internal threads inside of syringe body 126 further cause a threaded lead screw 130 to rotate. Lead screw 130 is prevented from rotating by a spring 132, such that the rotation of lead screw 130 is translated into linear movement of a plunger 134 having a threaded portion within lead screw 130. Linear movement of plunger 134 causes medicament to be expelled from the reservoir 104 and delivered to the user through, e.g., a cannula (not pictured). Delivery mechanism can further include an encoder 122 having a plurality of teeth that rotates when drive gear 116 is rotated and can be used to track the accuracy of delivery with pump 100 and other system conditions.

Flexible member 128 can also provide the spring force required to reset the drive mechanism after actuation. When the SMA wire 108 is de-energized, the wire 108 returns to its initial, longer length and the force pulling down on pulley 112a and flexible member 128 is released, which causes the flexible member to flex back up to its original position. An anti-back-drive tooth 120 of delivery mechanism frame 124 can interface with teeth to prevent the drive gear 116 from rotating in the opposite direction and back-driving during this reset phase. Delivery mechanism frame 124 can also function to retain the pulleys around which the SMA wire is routed in a plurality of pulley supports 132. Delivery mechanism frame 124 can further include a one-way crimp stop 111a that prevents the wire crimp 110a from moving in one direction when a smaller pulse is desired, but allows the crimp to move in the other direction when the larger pulse is desired, as will be discussed in more detail below.

Patch pump can be actuated to give a consistent volume of fluid per electrical pulse at regular intervals such as every five minutes such that one or more pulses is required to deliver the desired volume of medicament each interval. Often the desired volume at a given time is more than a single pulse so embodiments herein are provided with an optionally larger pulse size so that the number of actuations can be reduced to limit the drain on the batteries, but a small pulse size is still enabled so that precision of delivery can be maintained.

Referring to FIGS. 3A-3C, embodiments disclosed herein provide two distinct electrical paths for the SMA wire in order to provide two different dispense sizes with a single continuous length of wire. FIG. 3A depicts a first, shorter, electrical path with the SMA wire 108 extending from crimp connector 110a around pulleys 112 and to crimp connector 110b. FIG. 3B depicts a second, longer electrical path with the SMA wire 108 that includes the length between crimp connector 110a and crimp connector 110b, and also includes an additional length from crimp connector 110b to crimp connector 110c. FIG. 3C depicts only the crimp connectors and the SMA wire for sake of clarity, including the shorter SMA wire portion 108a and the longer portion including both portion 108a and 108b. In embodiment, the wire portions 108a, 108b are generally equal in length such that the longer path including both wire portions is approximately double the shorter path.

The different electrical paths can be selectively actuated by energizing respective electrical contacts at the corresponding crimp connectors. Actuation of the shorter electrical path of FIG. 3A results in a smaller rotation of drive gear 116 and a smaller quantity of medicament delivered, whereas actuation of the longer electrical path of FIG. 3B results in a larger rotation of drive gear and accordingly a larger quantity of medicament delivered. In embodiments, if the medicament is regular concentration (U100) insulin the amount of medicament delivered for the smaller pulse size can be between, for example, 0.025 to 0.075 units/pulse and between 0.050 and 0.15 units/pulse for the larger pulse size. In embodiments using ultra-concentrated insulin (U-200), these amounts double the amounts/ranges above given the higher concentration in the delivered amount of fluid. As noted above, when shorter path 108a is actuated, crimp stop 111a interfaces with crimp connector 110a to prevent movement in one direction (i.e., upward in the orientation of the figures) to cause the SMA wire 118 to pull down on the flexible member 128 to rotate the drive gear 116. When the longer path 108b is actuated, crimp connector 110a is able to move in the opposite direction (i.e., downward) to pull down on pulley 112b and flange 113 (see FIG. 2E), which further shortens the SMA wire 108 to increase the force on the flexible member 128 and rotation of the drive gear 116.

Pump 100 therefore allows for multiple pulse and delivery sizes in an SMA wire driven infusion pump using a single length of SMA wire. The ability to provide a second, larger dispense size results in fewer total actuations over the life of the pump which improves battery life and increases bolus rate of delivery. In one embodiment, use of two delivery sizes provides an approximately 40% increase in efficiency over use of a single delivery size. Use of a single length of SMA wire is also advantageous for reducing cost and complexity. In embodiments, patch pump can be assembled modularly to enable the pump to be assembled and tested prior to mating the PCBA which is desirable for manufacturability. Although described herein as having two different dispense sizes, it should be understood that embodiments contemplated herein could include more than two electrical paths that would define more than two dispense sizes.

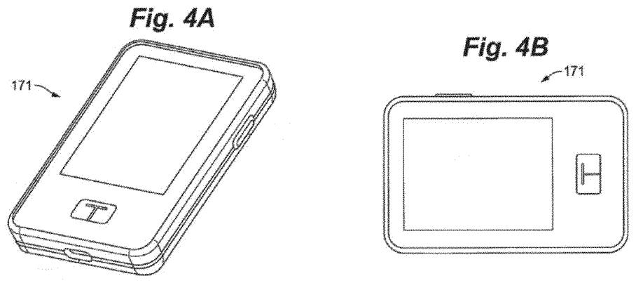

Referring to FIGS. 4A-4B, one or more remote control devices 170, 171 can be used to communicate with the processor of patch pump 100 to control delivery of medicament and transfer data with pump 100 via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device, such as a smart phone (not depicted) executing a software application for control of the pump, a dedicated remote controller 171 (as depicted in FIGS. 4A-4B), a wearable electronic watch or electronic health or fitness monitor or personal digital assistant (PDA), etc., or a tablet, laptop or personal computer. Such communications between (and among) the one or more remote control devices and pump 100 may be one-way or two-way for, e.g., effective transfer of data among the devices and the pump, control of pump operations, updating software on the devices and/or pump, and allowing pump-related data to be viewed on the devices and/or pump.

Embodiments of the present disclosure include components capable of and methods using wired and wireless transmission and receipt of signals for exchange of information and commands between and among any of the components as described herein, including, e.g., between a pump and a smartphone; among a pump, a CGM and a smartphone; between a dedicated remote controller and a pump; among a dedicated remote controller, a CGM and a pump; among a dedicated remote controller, a BGM and a pump, and other combinations as would be contemplated by those of skill in the art.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; and 11,305,057 and commonly owned U.S. Patent Publication Nos. 2009/0287180; 2012/0123230; 2013/0053816; 2014/0276423; 2014/0276569; 2014/0276570; 2018/0071454; 2019/0240398; 2019/0307952; 2020/0206420; 2020/0261649; 2020/0329433; 2020/0368430; 2020/0372995; 2021/0001044; 2021/0113766; 2021/0154405; 2021/0353857; 2022/0062553; 2022/0139522 and 2022/0223250 and commonly owned U.S. patent applications Ser. Nos. 17/368,968; 17/587,412; 17/587,434; 17/587,468; 17/677,621; 17/729,464; 17/732,208; 17/878,681; and 17/879,959.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A user-wearable infusion pump, comprising:
a housing;
a reservoir configured to contain a medicament disposed in the housing;
a delivery mechanism configured to deliver medicament from the reservoir to a user;
an actuation wire configured to be energized to actuate the delivery mechanism to deliver medicament from the reservoir to a user.

2. The user-wearable infusion pump of claim 1, wherein the actuation wire defines two electrical paths that are separately energizable.

3. The user-wearable infusion pump of claim 2, wherein the two electrical paths are configured to provide two different predetermined medicament dispense sizes with the delivery mechanism.

4. The user-wearable infusion pump of claim 2, wherein each electrical path is defined by corresponding electrical contacts that are energized to actuate the delivery mechanism.

5. The user-wearable infusion pump of claim 2, wherein the actuation wire is a single continuous wire.

6. The user-wearable infusion pump of claim 1, wherein the actuation wire comprises a shape memory material such that energization of the actuation wire causes the actuation wire to shorten to actuate the delivery mechanism.

7. The user-wearable infusion pump of claim 1, wherein the delivery mechanism comprises a drive gear configured to be rotated to actuate the delivery mechanism.

8. The user-wearable infusion pump of claim 7, wherein the delivery mechanism further comprises a frame disposed around the drive gear, the frame including a flexible member having a drive tooth configured to interface with the drive gear to rotate the drive gear.

9. The user-wearable infusion pump of claim 8, wherein the actuation wire is configured to pull the flexible member down to causes the drive tooth to rotate the drive gear a predetermined amount.

10. The user-wearable infusion pump of claim 7, wherein the delivery mechanism further includes an encoder having a plurality of teeth that is rotated when the drive gear rotates to track accuracy of medicament delivery.

11. A user-wearable infusion pump, comprising:
a housing;
a reservoir configured to contain a medicament disposed in the housing; and
a delivery mechanism configured to deliver medicament from the reservoir to a user, wherein the delivery mechanism is configured to provide two different predetermined medicament dispense sizes.

12. The user-wearable infusion pump of claim 11, further comprising an actuation wire configured to be energized to actuate the delivery mechanism to deliver medicament from the reservoir to a user.

13. The user-wearable infusion pump of claim 12, wherein the actuation wire defines two electrical paths corresponding to the two different predetermined medicament dispense sizes.

14. The user-wearable infusion pump of claim 13, wherein each electrical path is defined by corresponding electrical contacts that are energized to actuate the delivery mechanism.

15. The user-wearable infusion pump of claim 12, wherein the actuation wire is a single continuous wire.

16. The user-wearable infusion pump of claim 12, wherein the actuation wire comprises a shape memory material such that energization of the actuation wire causes the actuation wire to shorten to actuate the delivery mechanism.

17. The user-wearable infusion pump of claim 11, wherein the delivery mechanism comprises a drive gear configured to be rotated to actuate the delivery mechanism.

18. The user-wearable infusion pump of claim 17, wherein the delivery mechanism further comprises a frame disposed around the drive gear, the frame including a flexible member having a drive tooth configured to interface with the drive gear to rotate the drive gear.

19. The user-wearable infusion pump of claim 18, wherein the flexible member is configured to be pulled down to cause the drive tooth to rotate the drive gear a predetermined amount.

20. The user-wearable infusion pump of claim 17, wherein the delivery mechanism further includes an encoder having a plurality of teeth that is rotated when the drive gear rotates to track accuracy of medicament delivery.
